(19) United States
(12) Patent Application Publication
(10) Pub. No.: US 2011/0060248 A1
(43) Pub. Date: Mar. 10, 2011

(54) PHYSICAL CONFIGURATION DETECTOR, PHYSICAL CONFIGURATION DETECTING PROGRAM, AND PHYSICAL CONFIGURATION DETECTING METHOD

(76) Inventors: Tomotoshi Ishida, Hitachinaka (JP); Yushi Sakamoto, Yokohama (JP)

(21) Appl. No.: 12/866,721

(22) PCT Filed: Mar. 18, 2009
(86) PCT No.: PCT/JP2009/055346

§ 371 Date: Nov. 19, 2010

(30) Foreign Application Priority Data
Mar. 18, 2008 (JP) 2008-069474

(51) Int. Cl. A61B 5/103

(52) U.S. Cl. 600/587

(57) ABSTRACT

Disclosed is a physical configuration detector, a physical configuration detecting program, and a physical configuration detecting method, which can detect the physical configuration of an object, regardless of whether the object is in motion or not, and which can also decrease the amount of work necessary to prepare for dictionaries and the like. A physical configuration detector comprises: a sensor data acquisition unit (121) that acquires sensor data from directional sensors (10) attached to various points on the body a worker; a physical configuration calculator (122) that uses sensor data (113) to calculate the physical configuration which indicate the direction s the various points face; a positional data generator (123) that generates position data for the various points within a space, by using pre-stored shape data (111) and the physical configuration data (114) of the various points; a two-dimensional image generator (124) that generates two-dimensional image data indicating the various points by using position data (115) and shape data (111) for the various points; and a display controller (128) that displays the two-dimensional image data for the various points on a display (103).

### INCORPORATION BY REFERENCE

This application claims priority based on a Japanese patent application, No. 2008-069474 filed on Mar. 18, 2008, the entire contents of which are incorporated herein by reference.

### TECHNICAL FIELD

The present invention relates to a technique of grasping a posture of an object on the basis of outputs from directional sensors for detecting directions in space, the directional sensors being attached to some of target parts of the object.

### BACKGROUND ART

As a technique of grasping a posture of a human being or a device, there is a technique described in the following Patent Document 1, for example.

The technique described in Patent Document 1 involves attaching acceleration sensors to body parts of a human being as a target object in order to grasp motions of the body parts of that human being by using outputs from the acceleration sensors. First, according to this technique, outputs from the acceleration sensors at each type of motion are subjected to frequency analysis and output intensity of each frequency is obtained. Thus, a relation between a motion and respective output intensities of frequencies is investigated. Further, according to this technique, a typical pattern of output intensities of frequencies for each type of motion is stored in a dictionary. And, a motion of a human being is identified by making frequency analysis of actual outputs from acceleration sensors attached to the body parts of the human being and by judging which pattern the analysis result corresponds to.

Patent Document 1: Japanese Patent No. 3570163

### DISCLOSURE OF THE INVENTION

However, according to the technique described in Patent Document 1, it is difficult to grasp a posture of a human being if he continues to be in a stationary state such as a state of stooping down or a state of sitting in a chair. Further, it is very laborious to prepare the dictionary, and a large number of man-hour is required for preparing the dictionary in order to grasp many types of motions and in order to grasp combined motions each consisting of many motions.

Noting these problems of the conventional technique, an object of the present invention is to make it possible to grasp a posture of an object whether the object is in motion or in a stationary state, while reducing man-hour required for preparation such as creation of a dictionary.

To solve the above problems, according to the present invention:

a directional sensor for detecting a direction in space is attached to some target part among a plurality of target parts of a target object;

an output value from the directional sensor is acquired;

posture data indicating a direction of the target part, to which the directional sensor is attached, with reference to reference axes that are directed in previously-determined directions are calculated by using the output value from the directional sensor;

positional data of the target part in space are generated by using previously-stored shape data of the target part and the previously-calculated posture data of the target part, and by obtaining positional data in space of at least two representative points in the target part indicated in the shape data, with reference to a connecting point with another target part connected with the target part in question;

two-dimensional image data indicating the target part are generated by using the positional data in space of the target part and the previously-stored shape data of the target part stored; and

a two-dimensional image of the target part is outputted on a basis of the two-dimensional image data of the target part.

According to the present invention, it is possible to grasp the posture of a target object whether the target object is in motion or in a stationary state. Further, according to the present invention, by previously acquiring shape data of a target body part, it is possible to grasp the posture of this target body part. And thus, man-hour required for preparation (such as creation of a dictionary for grasping postures) can be diminished very much.

### BRIEF DESCRIPTION OF DRAWINGS

FIG. 1 is a block diagram showing a posture management system in a first embodiment of the present invention;

FIG. 2 is a block diagram showing a directional sensor in the first embodiment of the present invention;

FIG. 3 is an explanatory diagram showing a worker in a schematic illustration according to the first embodiment of the present invention;

FIG. 4 is an explanatory diagram showing data structure of shape data in the first embodiment of the present invention;

FIG. 5 is an explanatory diagram showing a relation between a common coordinate system and a local coordinate system in the first embodiment of the present invention;

FIG. 6 is an explanatory diagram showing data structure of motion evaluation rule in the first embodiment of the present invention;

FIG. 7 is an explanatory diagram showing data structure of sensor data in the first embodiment of the present invention;

FIG. 8 is an explanatory diagram showing data structure of posture data in the first embodiment of the present invention;

FIG. 9 is an explanatory diagram showing data structure of positional data in the first embodiment of the present invention;

FIG. 10 is a flowchart showing operation of a posture grasping apparatus in the first embodiment of the present invention;

FIG. 11 is a flowchart showing the detailed processing in the step 30 of the flowchart of FIG. 10;

FIG. 12 is an illustration for explaining an example of an output screen in the first embodiment of the present invention;

FIG. 13 is a block diagram showing a posture grasping system in a second embodiment of the present invention;

FIG. 14 is an explanatory diagram showing data structure of trailing relation data in the second embodiment of the present invention;

FIG. 15 is a block diagram showing a posture grasping system in a third embodiment of the present invention;

FIG. 16 is an explanatory diagram showing data structure of sensor data in the third embodiment of the present invention;

FIG. 17 is an explanatory diagram showing data structure of second positional data and a method of generating the second positional data in the third embodiment of the present invention;

FIG. 18 is a flowchart showing operation of a posture grasping apparatus in the third embodiment of the present invention; and

FIG. 19 is an illustration for explaining an example of an output screen in the third embodiment of the present invention.

### SYMBOLS

10: directional sensor; 11: acceleration sensor; 12: magnetic sensor; 100, 100a, 100b: posture grasping apparatuses; 103: display; 110: storage unit; 111: shape data; 112: motion evaluation rule; 113, 113B: sensor data; 114: posture data; 115: positional data; 116, 116B: two-dimensional image data; 117: motion evaluation data; 118: work time data; 119: trailing relation data; 120: CPU; 121: sensor data acquisition unit; 122, 122a: posture data calculation unit; 123: positional data generation unit; 124, 124b: two-dimensional image data generation unit; 125: motion evaluation data generation unit; 127: input control unit; 128: display control unit; 129: second positional data generation unit; 131: memory; 132: communication unit; and 141: second positional data

### BEST MODE FOR CARRYING OUT THE INVENTION

In the following, embodiments of posture grasping system according to the present invention will be described referring to the drawings.

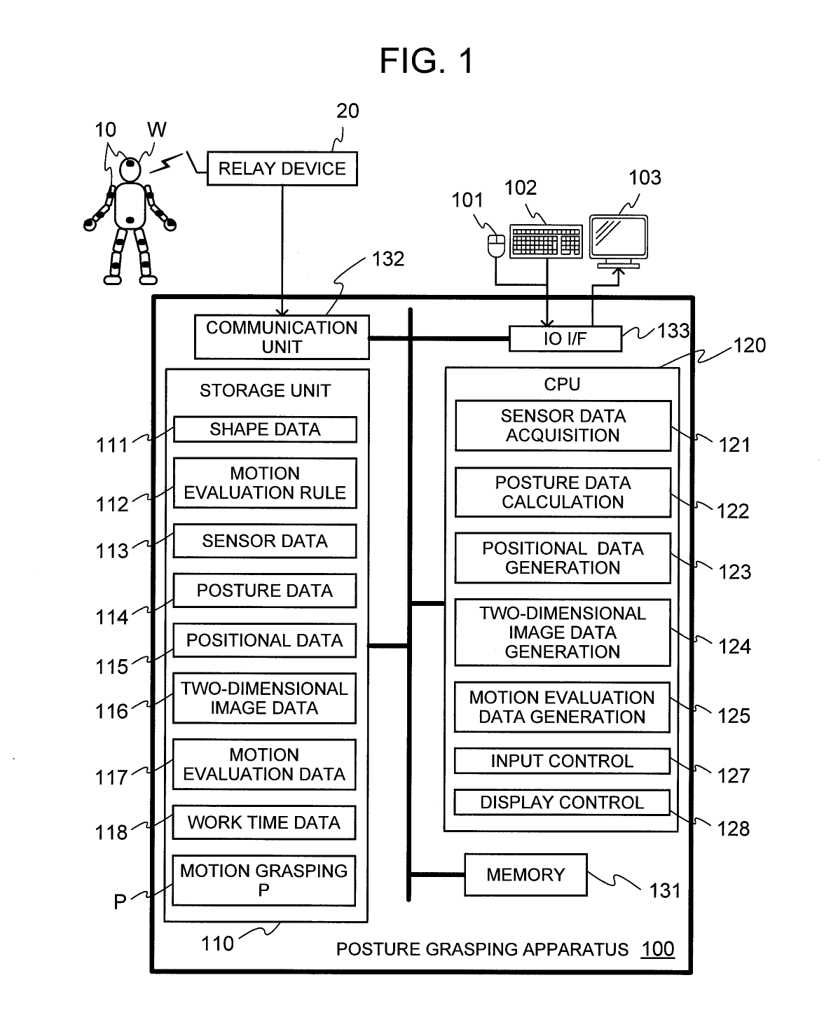

First, a first embodiment of posture grasping system will be described referring to FIGS. 1-12.

As shown in FIG. 1, the posture grasping system of the present embodiment comprises: a plurality of directional sensors 10 attached to a worker W as an object of posture grasping; and a posture grasping apparatus 100 for grasping a posture of the worker W on the basis of outputs from the directional sensors 10.

The posture grasping apparatus 100 is a computer comprising: a mouse 101 and a keyboard 102 as input units; a display 103 as an output unit; a storage unit 110 such as a hard disk drive or a memory; a CPU 120 for executing various operations; a memory 131 as a work area for the CPU 120; a communication unit 132 for communicating with the outside; and an I/O interface circuit 133 as an interface circuit for input and output devices.

The communication unit 132 can receive sensor output values from the directional sensors 10 via a radio relay device 20.

The storage unit 110 stores shape data 111 concerning body parts of the worker W, a motion evaluation rule 112 as a rule for evaluating a motion of the worker W, and a motion grasping program P, in advance. In addition, the storage unit 110 stores an OS, a communication program, and so on, although not shown. Further, in the course of execution of the motion grasping program P, the storage unit 110 stores sensor data 113, posture data 114 indicating body parts' directions obtained on the basis of the sensor data 113, positional data 115 indicating positional coordinate values of representative points of the body parts, two-dimensional image data 116 for displaying the body parts on the display 103, motion evaluation data 117 i.e. motion levels of the body parts, and work time data 118 of the worker W.

The CPU 120 functionally comprises (i.e. functions as): a sensor data acquisition unit 121 for acquiring the sensor data from the directional sensors 10 through the communication unit 132; a posture data calculation unit 122 for calculating the posture data that indicate body parts' directions on the basis of the sensor data; a positional data generation unit 124 for generating positional data that indicate positional coordinate values of representative points of the body parts; a two-dimensional image data generation unit 124 for transforming body parts' coordinate data expressed as tree-dimensional coordinate values, into two-dimensional coordinate values; a motion evaluation data generation unit 125 for generating the motion evaluation data as motion levels of the body parts; an input control unit 127 for input control of the input units 101 and 102; and a display control unit 128 for controlling the display 103. Each of these functional control units functions when the CPU 120 executes the motion grasping program P stored in the storage unit 110. In addition, the sensor data acquisition unit 121 functions when the motion grasping program P is executed under the OS and the communication program. And the input control unit 127 and the display control unit 128 function when the motion grasping program P is executed under the OS.

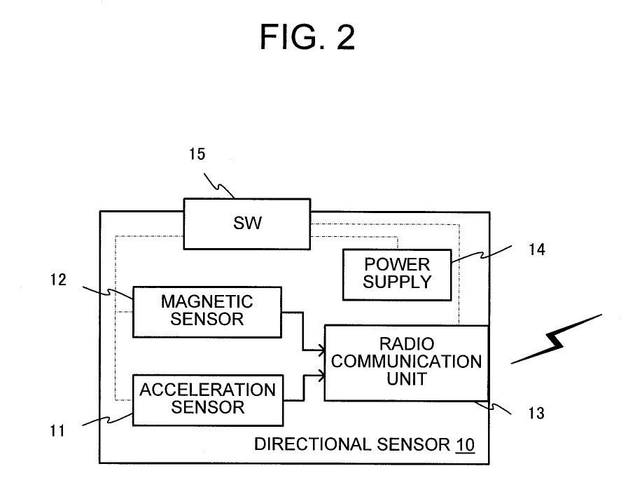

As shown in FIG. 2, each of the directional sensors 10 comprises: an acceleration sensor 11 that outputs values concerning directions of mutually-perpendicular three axes; a magnetic sensor 12 that outputs values concerning directions of mutually-perpendicular three axes; a radio communication unit 13 that wirelessly transmits the outputs from the sensors 11 and 12; a power supply 14 for these components; and a switch 15 for activating these components. Here, the acceleration sensor 11 and the magnetic sensor 12 are set such that their orthogonal coordinate systems have the same directions of axes. In the present embodiment, the acceleration sensor 11 and the magnetic sensor 12 are set in this way to have the same directions of axes of their orthogonal coordinate systems, because it simplifies calculation for obtaining the posture data from these sensor data. It is not necessary that the sensors 11 and 12 have the same directions of axes of their orthogonal coordinate systems.

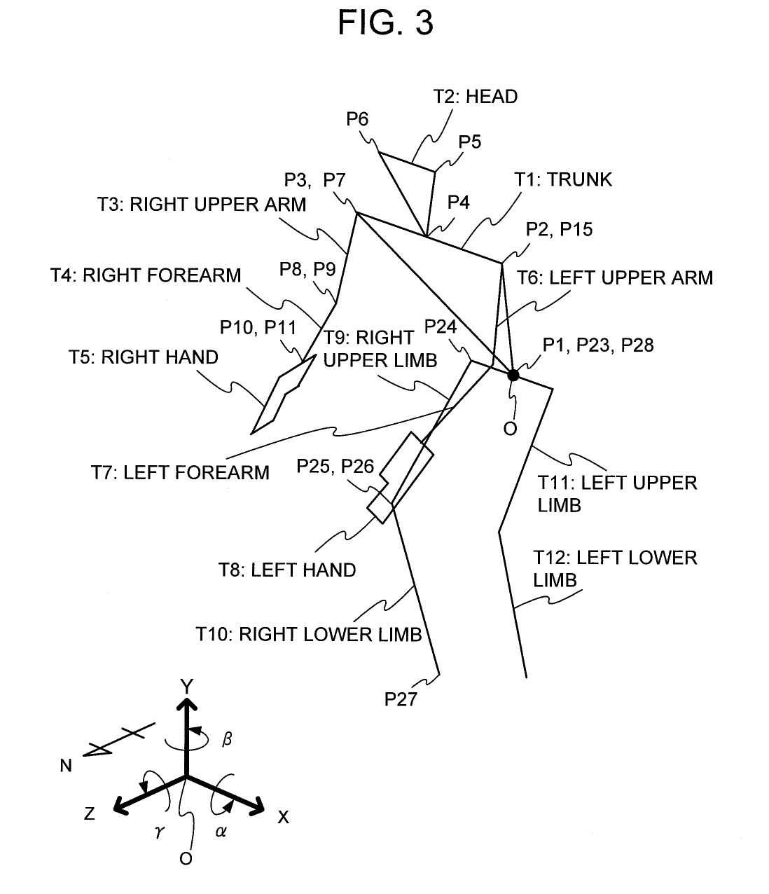

The shape data 111, which have been previously stored in the storage unit 110, exist for each motion part of the worker. As shown in FIG. 3, in this embodiment, the motion parts of the worker are defined as a trunk T1, a head T2, a right upper arm T3, a right forearm T4, a right hand T5, a left upper arm T6, a left forearm T7, a left hand T8, a right upper limb T9, a right lower limb T10, a left lower limb T11, and a left lower limb T12. Although the worker's body is divided into the twelve motion parts in the present embodiment, the body may be divided into more body parts including a neck and the like. Or, an upper arm and a forearm can be taken as a unified body part.

In the present embodiment, to express the body parts in a simplified manner, the trunk T1 and the head T2 are each expressed as an isosceles triangle, and the upper arms T3, T6, the forearms T4, T7 and the like are each expressed schematically as a line segment. Here, some points in an outline of each body part are taken as representative points, and a shape of each body part is defined by connecting such representative points with a line segment. Here, the shape of any part is extremely simplified. However, to approximate a shape to the actual one of the worker, a complex shape may be employed. For example, the trunk and the head may be expressed respectively as three-dimensional shapes.

In FIG. 3, a common coordinate system XYZ is used for expressing the worker as a whole, the vertical direction being expressed by the X-axis, the north direction by the Z-axis, and the direction perpendicular to the Y- and Z-axes by the X-axis. A representative point indicating the loin of the trunk T1 is expressed by the origin O. Further, directions around the axes are expressed by α, β and γ, respectively.

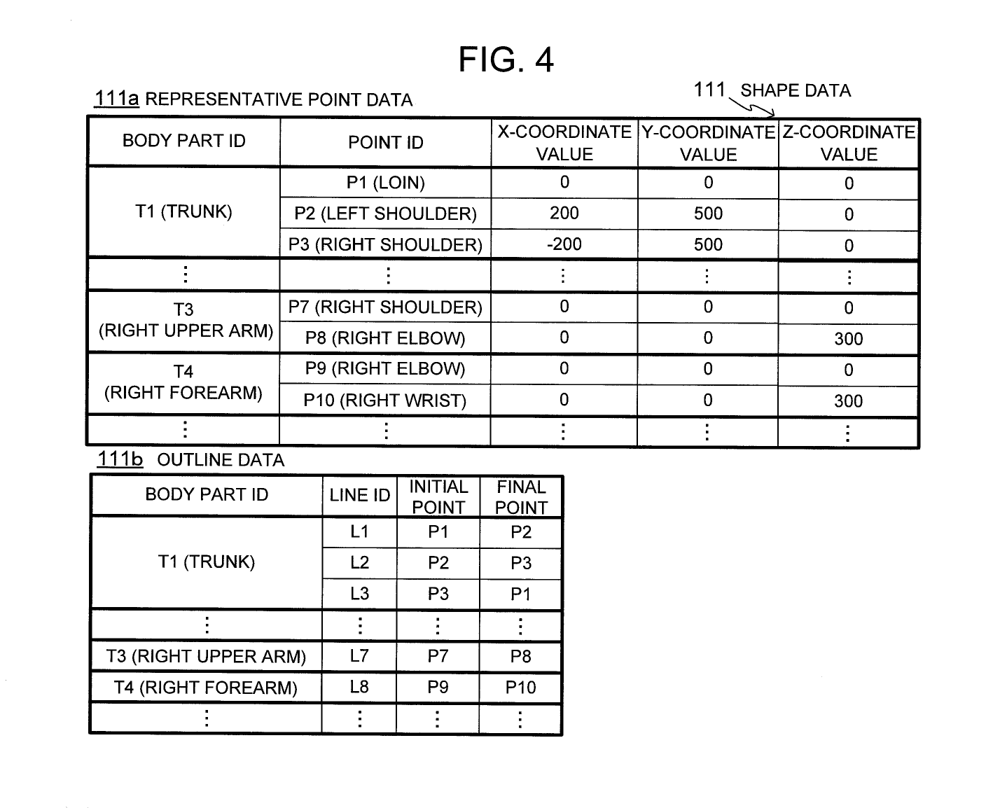

As shown in FIG. 4, shape data 111 of the body parts comprise representative point data 111a and outline data 111b, the representative point data 111a indicating three-dimensional coordinate values of the representative points of the body parts, and the outline data 111b indicating how the representative points are connected to form the outline of each body part.

The representative point data 111a of each body part comprise a body part ID, representative point IDs, and X-, Y-, and Z-coordinate values of each representative point. For example, the representative point data of the trunk comprise the ID “T1” of the trunk, the IDs “P1”, “P2” and “P3” of three representative points of the trunk, and coordinate values of these representative points. And, the representative point data of the right forearm comprise the ID “T4” of the right forearm, the IDs “P9” and “P10” of two representative points of the right forearm, and coordinate values of these representative points.

The outline data 111b of each body part comprises the body part ID, line IDs of lines expressing the outline of the body part, IDs of initial points of these lines, and IDs of final points of these lines. For example, as for the trunk, it is shown that the trunk is expressed by three lines L1, L2 and L3, the line L1 having the initial point P1 and the final point P2, the line L2 the initial point P2 and the final point P3, and the line L3 the initial point P3 and the final point P1.

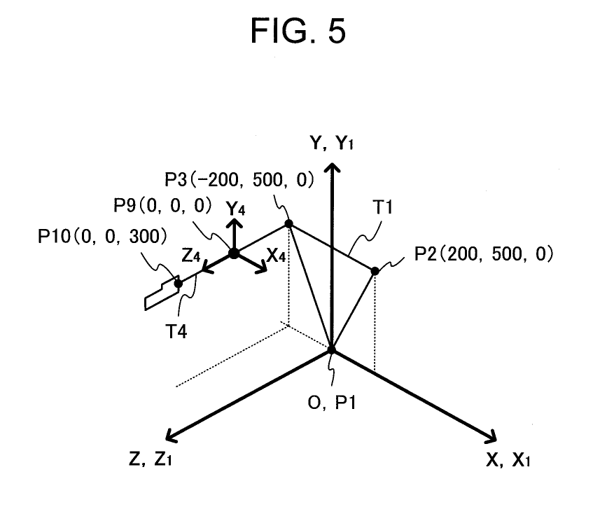

In the present embodiment, the coordinate values of a representative point of each body part are expressed in a local coordinate system for each body part. As shown in FIG. 5, the origin of the local coordinate system of each body part is located at a representative point whose ID has the least number among the representative points of the body part in question. For example, the origin of the local coordinate system X₁Y₁Z₁ of the trunk T1 is located at the representative point P1. And, the origin of the local coordinate system X₄Y₄Z₄ of the right forearm T4 is located at the representative point P9. Further, the X-, Y- and Z-axes of each local coordinate system are respectively parallel to the X-, Y- and Z-axes of the common coordinate system XYZ described referring to FIG. 3. This parallelism of the X-, Y- and Z-axes of each local coordinate to the X-, Y- and Z-axes of the common coordinate system XYZ is employed because transformation of a local coordinate system into the common coordinate system does not require rotational processing. It is not necessary that the X-, Y- and Z-axes of each local coordinate system are parallel to the X-, Y- and Z-axes of the common coordinate system XYZ. By locating the origin O of the common coordinate system XYZ at the representative point P1 of the trunk, the common coordinate system XYZ is identical with the trunk local coordinate system X₁Y₁Z₁. Thus, in the present embodiment, the representative point P1 becomes a reference position in transformation of coordinate values in each local coordinate system into ones in the common coordinate system.

Coordinate values of any representative point in each body part are indicated as coordinate values in its local coordinate system in the state of a reference posture. For example, as for the trunk T1, a reference posture is defined as a posture in which all the three representative points P1, P2 and P3 all located in the X₁Y₁ plane of the local coordinate system X₁Y₁Z₁ and the Y₁ coordinate values of the representative points P2 and P3 are the same value. The coordinate values of the representative points in this reference posture constitute the representative point data 111a of the trunk T1. As for the forearm T4, a reference posture is defined as a posture in which both the two representative points P9 and P10 are located on the Z₄-axis of the local coordinate system X₄Y₄Z₄. And, the coordinate values of the representative points in this reference posture constitute the representative point data 111a of the forearm T4.

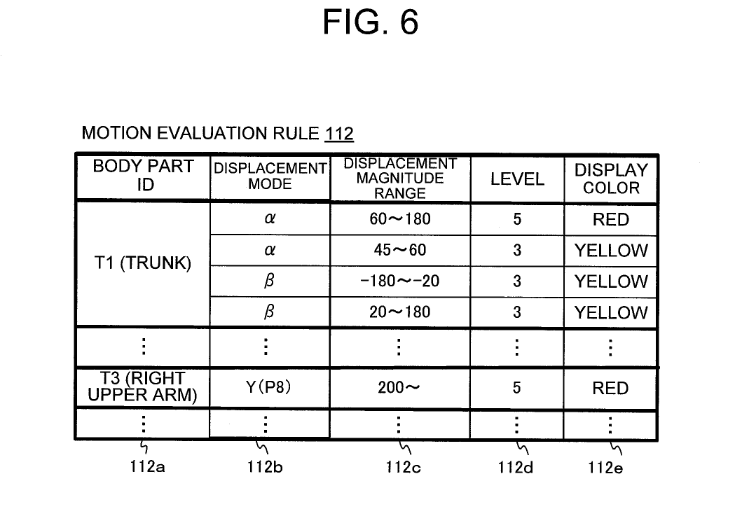

As shown in FIG. 6, the motion evaluation rule 112 previously stored in the storage unit 110 is expressed in a table form. This table has: a body part ID field 112a for storing a body part ID; a displacement mode field 112b for storing a displacement mode; a displacement magnitude range field 112c for storing a displacement magnitude range; a level field 112d for storing a motion level of a displacement magnitude belonging to the displacement magnitude range; and a display color field 112e for storing a display color used for indicating the level. Here, a displacement mode stored in the displacement mode field 112b indicates a direction of displacement.

In this motion evaluation rule 112, as for the trunk T1 for example, when the angular displacement in the α direction is within the range of 60°-180° or 45°-60°, the motion level is “5” or “3”, respectively. And, when the motion level “5” is displayed, display in “Red” is specified, while the motion level “3” is displayed, display in “Yellow” is specified. Further, as for the right upper arm T3, the table shows that the motion level is “5” when the displacement magnitude in the Y-axis direction of the representative point P8 in the Y direction is 200 or more, and its display color is “Red”. Here, the displacement magnitude is one relative to the above-mentioned reference posture of the body part in question.

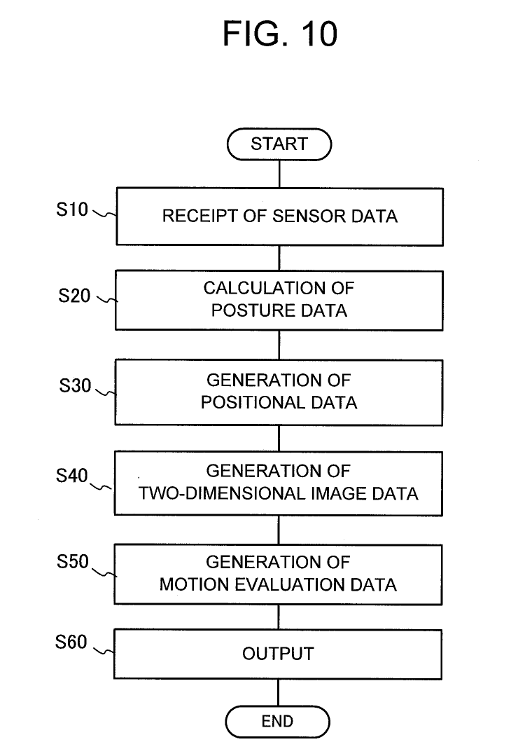

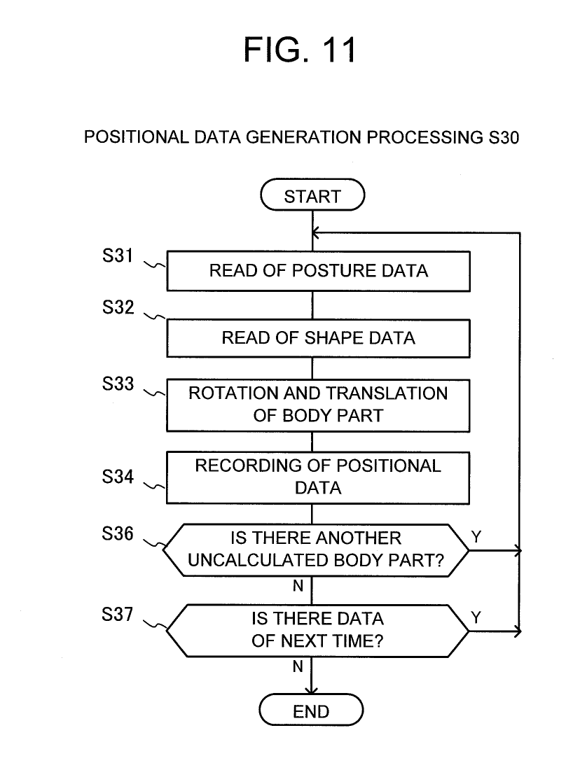

Next, referring to flowcharts shown in FIGS. 10 and 11, operation of the posture grasping apparatus 100 of the present embodiment will be described.

When the worker attaches a directional sensor 10 to his body part and turns on the switch 15 (FIG. 2) of this directional sensor 10, data measured by the directional sensor 10 is transmitted to the posture grasping apparatus 100 through a relay device 20.

When the sensor data acquisition unit 121 of the posture grasping apparatus 100 receives the data from the directional sensor 10 through the communication unit 132, the sensor data acquisition unit 121 stores the data as sensor data 113 in the storage unit 110 (S10).

When the sensor data acquisition unit 121 receives data from a plurality of directional sensors 10 attached to a worker, the sensor data acquisition unit 121 does not store these data in the storage unit 110 immediately. Only when it is confirmed that data have been received from all the directional sensors 10 attached to the worker, the sensor data acquisition unit 121 stores the data from the directional sensors 10 in the storage unit 110 from that point of time. If data cannot be received from any directional sensor 10 among all the directional sensors 10 attached to a worker, the sensor data acquisition unit 121 does not store the data that have been received at this point of time from directional sensors 10 in the storage unit 110. In other words, only when there are data received from all the directional sensors 10 attached to a worker, the data are stored in the storage unit 110.

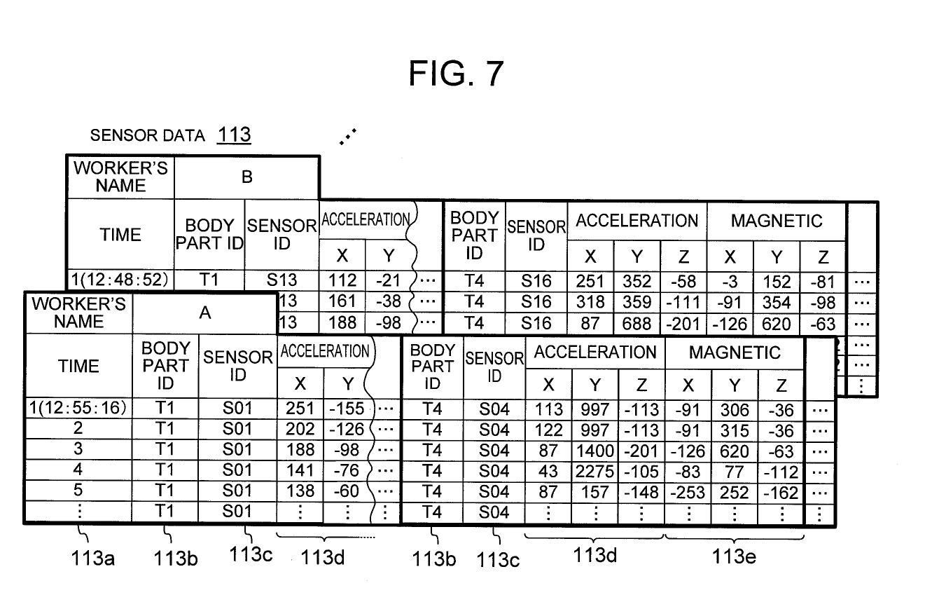

As shown in FIG. 7, the sensor data 113 stored in the storage unit 110 are expressed in the form of a table, and such a table exists for each of workers A, B, and so on. Each table has: a time field 113a for storing a receipt time of data; a body part ID field 113b for storing a body part ID; a sensor ID field 113c for storing an ID of a directional sensor attached to the body part; an acceleration sensor data field 113d for storing X, Y and Z values from the acceleration sensor included in the directional sensor 10; and a magnetic sensor data field 113e for storing X, Y and Z values from the magnetic sensor 12 included in the directional sensor 10. Although only data concerning the trunk T1 and the forearm T4 are seen in one record in the figure, in fact one record includes data concerning all the body parts of the worker. Further, the body part ID and the sensor ID are previously related with each other. That is to say, it is previously determined that a directional sensor 10 of ID “S01” is attached to the trunk T1 of the worker A, for example. Here, the X, Y and Z values from the sensors 11 and 12 are values in the respective coordinate systems of the sensors 11 and 12. However, the X-, Y- and Z-axes in the respective coordinate systems of the sensors 11 and 12 coincide with the X-, Y- and Z-axes in the local coordinate system of the body part in question if the body part to which the directional sensor 10 including these sensors 11 and 12 is attached is in its reference posture.

Next, the posture data calculation unit 122 of the posture grasping apparatus 100 calculates respective directions of the body parts on the basis of data shown in the sensor data 113 for each body part at each time, and stores, as posture data 114, data including thus-calculated direction data in the storage unit 113 (S20).

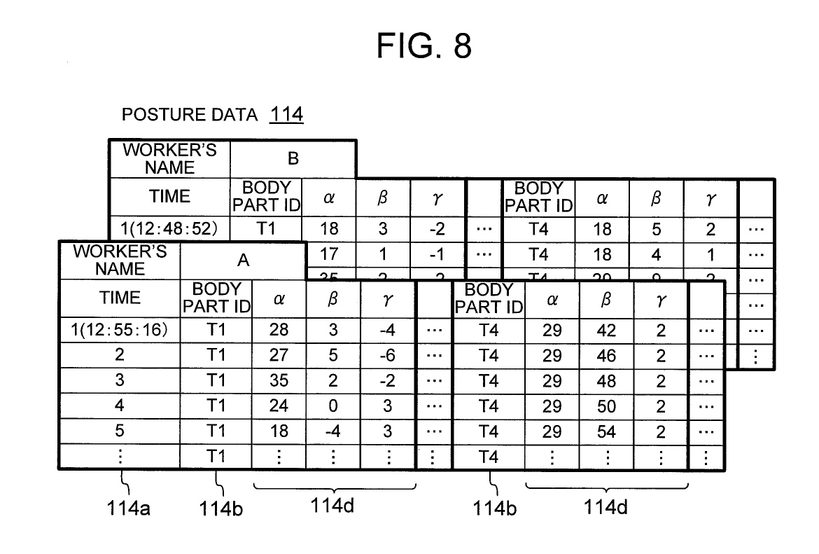

As shown in FIG. 8, the posture data 114 stored in the storage unit 110 is expressed in the form of a table, and such a table exists for each of the workers A, B, and so on. Each table has: a time field 114a for storing a receipt time of sensor data; a body part ID field 114b for storing a body part ID; and a direction data field 114d for storing angles in the α, β and γ directions of the body part in question. In this figure also, although only data concerning the trunk T1 and the forearm T4 are seen in one record, in fact one record includes data concerning all the body parts of the worker. Here, all α, β and γ are values in the local coordinate system.

Now, will be simply described a method of calculating data stored in the direction data field 114d from data stored in the acceleration sensor data field 113d and the magnetic sensor data field 113e in the sensor data 113.

For example, in the case where the right forearm T4 is made stationary in the reference posture, the acceleration in the direction of the Y-axis is −1G due to gravity, and the accelerations in the directions of the X- and Z-axes are 0. Thus, output from the acceleration sensor is (0, −1G, 0). When the right forearm is tilted in the α direction from this reference posture state, it causes changes in the values from the acceleration sensor 11 in the directions of the Y- and Z-axes. At this time, the value of α in the local coordinate system is obtained from the following equation using the values in the directions of the Y- and Z-axes from the acceleration sensor 11.

α=sin⁻¹(z/sqrt(z²+y²))

Similarly, when the right forearm T4 is tilted in the γ direction from the reference posture, the value of γ in the local coordinate system is obtained from the following equation using the values in the directions of the X- and Y-axes from the acceleration sensor 11.

γ=tan⁻¹(x/y)

Further, when the right forearm T4 is tilted in the β direction from the reference posture, the output values from the acceleration sensor 11 do not change but the values in the Z- and X-axes from the magnetic sensor 12 change. At this time, the value of β in the local coordinate system is obtained from the following equation using the values in the Z- and X-axes from the magnetic sensor 12.

β=sin⁻¹(x/sqrt(x²+z²))

Next, the positional data generation unit 123 of the posture grasping apparatus 100 obtains coordinate values of the representative points of the body parts in the common coordinate system by using the shape data 111 and the posture data 114 stored in the storage unit 111, and stores, as positional data 115, data including thus-obtained coordinate values in the storage unit 110 (S30).

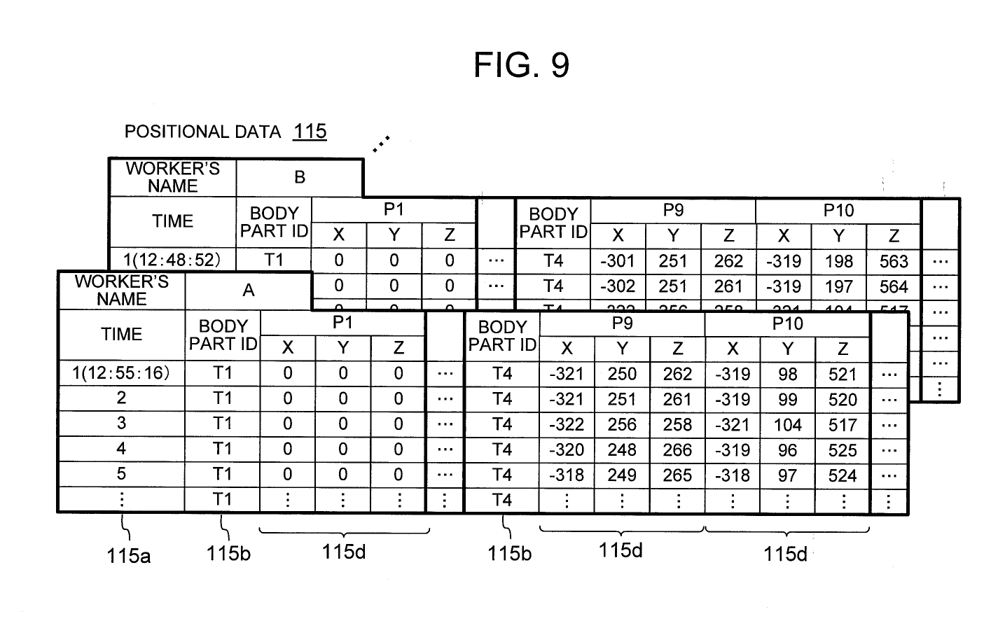

As shown in FIG. 9, also the positional data 115 stored in the storage unit 111 is expressed in the form of a table, and such a table exists for each of the workers A, B, and so on. Each table has: a time field 115a for storing a receipt time of sensor data; a body part ID field 115b for storing a body part ID; and a coordinate data field 115d for storing X-, Y- and Z-coordinate values in the common coordinate system of the representative points of the body part in question. In this figure also, although only data concerning the trunk T1 and the forearm T4 are seen in one record, in fact one record includes data concerning all the body parts of the worker. Further, the figure shows the coordinate values of the representative point P1 of the trunk T1. However, the representative point P1 is the origin O of the common coordinate system, and the coordinate values of the representative point P1 are always 0. Thus, the coordinate values of the representative point P1 may be omitted.

Now, a method of obtaining the coordinate values of a representative point of a body part will be described referring to the flowchart shown in FIG. 11.

First, among the posture data 114, the positional data generation unit 123 reads data in the first record (the record at the first receipt time) of the trunk T1 from the storage unit 110 (S31). Next, the positional generation unit 123 reads also the shape data 111 of the trunk T1 from the storage unit 110 (S32).

Next, the positional data generation unit 123 rotates the trunk T1 in the local coordinate system according to the posture data, and thereafter, translates the thus-rotated trunk T1 such that the origin P1 of the local coordinate system coincides with the origin of the common coordinate system, and obtains the coordinate values of the representative points of the trunk T1 in the common coordinate system at this point of time. In detail, first, the local coordinate values of the representative points P1, P2 and P3 of the trunk T1 are obtained by rotating the trunk T1 by the angles α, β and γ indicated in the posture data. Next, the coordinate values in the common coordinate system of the origin P1 of the local coordinate system are subtracted from these local coordinate values, to obtain the coordinate values in the common coordinate system (S33). Here, the local coordinate system of the trunk T1 and the common coordinate system coincide as described above, and thus it is not necessary to perform the translation processing in the case of the trunk T1.

Next, the positional data generation unit 123 stores the time data included in the posture data 114 in the time field 115a (FIG. 9) of the positional data 115, the ID (T1) of the trunk in the body part ID field 115b, and the coordinate values of the representative points of the trunk T1 in the coordinate data field 115d (S34).

Next, the positional data generation unit 123 judges whether there is a body part whose positional data have not been obtained among the body parts connected to a body part whose positional data have been obtained (S35).

If there is such a body part, the flow returns to the step 31 again, to read the posture data 114 in the first record (the record at the first receipt time) of this body part from the storage unit 110 (S31). Further, the shape data 111 of this body part are also read from the storage unit 110 (S32). Here, it is assumed for example that the shape data and the posture data of the right upper arm T3 connected to the trunk T1 are read.

Next, the positional data generation unit 123 rotates the right upper arm T3 in the local coordinate system according to the posture data, and then translates the thus-rotated right upper arm T3 such that the origin (the representative point) P7 of this local coordinate system coincides with the representative point P3 of the trunk T1 whose position has been already determined in the common coordinate system, to obtain the coordinate values of the representative points of the right upper arm T3 in the common coordinate system at this point of time (S33).

Further, as for the right forearm T3, the right forearm T4 is rotated in the local coordinate system according to the posture data, and thereafter the thus-rotated right forearm T4 is translated such that the origin (the representative point) P9 of this local coordinate system coincides with the representative point P8 of the right upper arm T3 whose position has been already determined in the common coordinate system. Then, the coordinate values in the common coordinate system of the representative points of the right forearm T4 are obtained at this time point.

Thereafter, the positional data generation unit 123 performs the processing in the steps 31-36 repeatedly until judging that there is no body part whose positional data have not been obtained among the body parts connected to a body part whose positional data have been obtained (S36). In this way, the coordinate values in the common coordinate system of a body part are obtained starting from the closest body part to the trunk T1.

Then, when the positional data generation unit 123 judges that there is no body part whose positional data have not been obtained among the body parts connected to a body part whose positional data have been obtained (S36), the positional data generation unit 123 judges whether there is a record of the trunk T1 at the next point of time in the posture data 114 (S37). If there is a record of the next point of time, the flow returns to the step 31 again, to obtain the positional data of the body parts at the next point of time. If it is judged that a record of the next time point does not exist, the positional data generation processing (S30) is ended.

Although it is not necessary to describe a detailed method of coordinate transformation relating to the above-mentioned rotation and translation of a body part in a three-dimensional space, detailed description of such a method is given in Computer Graphics; A Programming Approach, Japanese translation by KOHRIYAMA, Akira (Originally written by Steven Harrington), issued 1984 by McGraw-Hill, Inc, for example.

As shown in the flowchart of FIG. 10, when the positional data generation processing (S30) is finished, the two-dimensional image data generation unit 124 transforms the image data of the shape of the worker in the three-dimensional space into two-dimensional image data so that the image data of the shape of the worker can be displayed on the display 103 (S40). In this processing, the two-dimensional image data generation unit 124 uses one point in the common coordinate system as a point of sight, and generates a virtual projection plane oppositely to the point of sight with reference to a worker's image that is expressed by using the positional data 115 and the shape data 111 stored in the storage unit 110. Then, the worker's image is projected from the point of sight onto the virtual projection plane, and two-dimensional image data are obtained by determining coordinate values of the representative points of the body parts of the worker's image in the virtual projection plane.

Also, a method of transforming three-dimensional image data into two-dimensional image data is obvious and does not require detailed description. For example, Japanese Patent No. 3056297 describes such a method in detail.

Next, the motion evaluation data generation unit 125 generates the motion evaluation data 117 for each worker and work time data 118 for each worker, and stores the generated data 117 and 118 in the storage unit 110 (S50). The work time data 118 for each worker comprise a work start time and a work finish time for the worker in question. Among the times stored in the time field 113a of the sensor data 113 (FIG. 7) of a worker, the motion evaluation data generation unit 125 determines, as the work start time of the worker, the first time point in a time period during which data were successively received, and determines as the work finish time the last time point in this time period. A method generating the motion evaluation data 117 will be described later.

Next, the display control unit 128 displays the above processing results on the display 103 (S60).

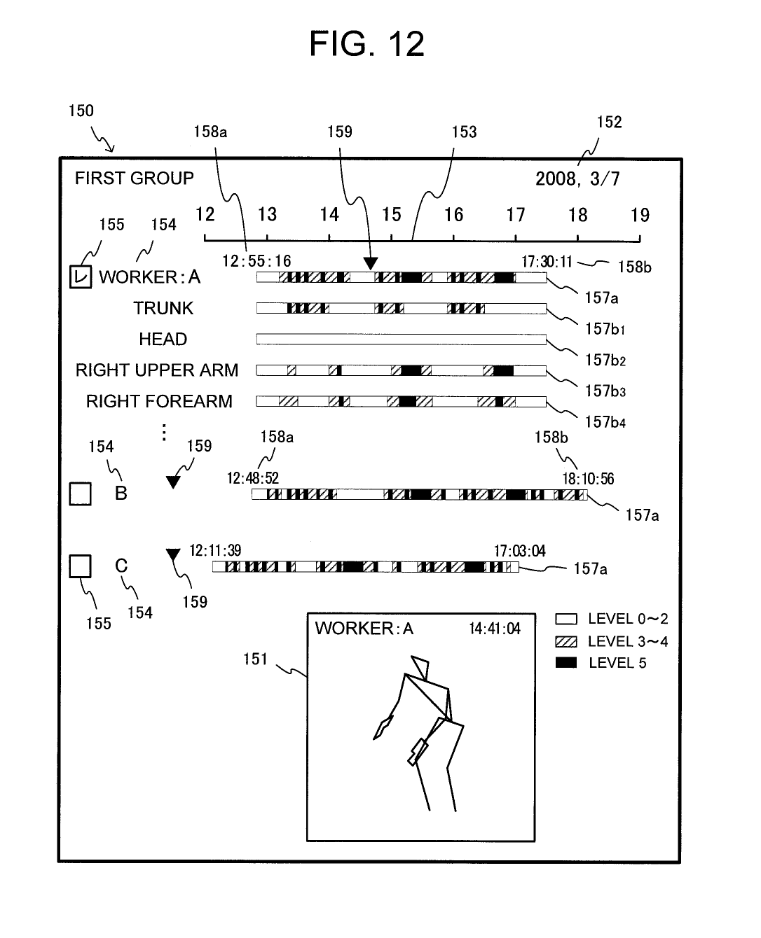

As shown in FIG. 12, an output screen 150 on the display 103 displays, first of all a date 152, a time scale 153 centering on working hours (13:00-17:00) of workers, workers' names 154, motion evaluation data expansion instruction boxes 155, integrated motion evaluation data 157a of the workers, work start times 158a of the workers, work finish times 158b of the workers, and time specifying marks 159.

When an operator wishes to know detailed motion evaluation data of a specific worker, not the integrated motion evaluation data 157a of the workers, the operator clicks the motion evaluation data expansion instruction box 155 displayed in front of the name of the worker in question. Then, the motion evaluation data 157b₁, 175b₂, 175b₃ and so on of the body parts of the worker in question are displayed.

As described above, motion evaluation data are generated by the motion evaluation data generation unit 125 in the step 50. The motion evaluation data generation unit 125 first refers to the motion evaluation rule 112 (FIG. 6) stored in the storage unit 110, and investigates a time period of displacement magnitude that enters a displacement magnitude range of each displacement mode of each body part. For example, as for the case where a body part is the trunk T1 and a displacement mode is the displacement in the α direction, a time period in which a displacement magnitude range is “60°-180°” (i.e. a time period of the level 5) is extracted from the posture data 114 (FIG. 8). Similarly, a time period in which a displacement magnitude range is “45°-60°” (i.e. a time period of the level 3) is extracted also. Further, also as for the case where a body part is the trunk T1 and a displacement mode is the displacement in the γ direction, time periods in which a displacement magnitude range is “−180°-−20°” or “20°-180°” (i.e. time periods of the level 3) are extracted from the posture data 114 (FIG. 8). Similarly, a time period in which a displacement magnitude range is “45°-60°” (i.e. a time period of the level 3) is extracted also. Then, motion level data, i.e. motion evaluation data concerning the trunk T1 at each time are generated. In so doing, since motion levels at each time are different for different displacement modes, the highest motion level at each time is determined as the motion level at that time.

Then, in the same way, the motion evaluation data generation unit 125 obtains a motion level at each time for each body part.

Next, the motion evaluation data generation unit 125 generates integrated motion evaluation data for the worker in question. In the integrated motion evaluation data, the highest motion level among the motion levels of the body parts of the worker at each time becomes an integrated motion level, i.e. the integrated motion evaluation data at that time.

The thus-generated motion evaluation data for the body parts and the thus-generated integrated motion evaluation data are stored as the motion evaluation data 117 of the worker in question in the storage unit 110. The display control unit 128 refers to the motion evaluation data 117 and displays in the output screen 150 the integrated motion evaluation data 157a for each worker, the motion evaluation data 157b₁, 157b₂, 157b₃, and so on for the body parts of specific worker. Here, in the motion evaluation data 157a, 157b₁, and so one, time periods of the level 5 and the level 3 are displayed in the colors stored in the display color field 122e (FIG. 6) of the motion evaluation rule 112.

When the operator sees the motion evaluation data 157a and so on of each worker and wishes to see the motion of a specific worker at a specific point of time, the operator moves the time specifying mark 159 to the time in question on the time scale 153. Then, a schematic dynamic state screen 151 of the worker after that point of time is displayed in the output screen 150. This dynamic state screen 151 is displayed by the display control unit 128 on the basis of the worker's two-dimensional image data 116 at each time which are stored in the storage unit 110. In this dynamic state screen 151, each body part of the worker is displayed in the color corresponding to its motion level. In this dynamic state screen 151, the representative point P1 of the trunk T1 of the worker becomes a fixed point, and other body parts move and rotate relatively. Accordingly, when the worker bends and stretches his legs, his loin (P1) does not go down and his feet go up, although his knees bend. Thus, if such dynamic display seems to be strange, it is possible to resolve elevation of the feet at the time of worker's bending and stretching, by translating the body parts in generation of the positional data in the step 30 such that the Y coordinate values of the feet becomes 0.

As described above, in the present embodiment, the posture data are generated on the basis of the sensor data from the directional sensors 10 whether any body part of the worker is in motion or in a stationary state, and a schematic image data of the worker are generated on the basis of the posture data. As a result, it is possible to grasp the posture of the body parts of the worker whether the worker is in motion or in a stationary state. Further, in the present embodiment, the posture of the body parts can be grasped by preparing the shape data 111 of the body parts in advance. Thus, man-hour required for preparation (such as creation of a dictionary for grasping postures) can be diminished very much.

Further, in the present embodiment, the motion evaluation level of each worker and the motion evaluation level of each body part of a designated worker are displayed at each time. Thus, it is possible to know which worker has a heavy workload at which time, and further which part of the worker has a heavy workload. Further, since also the work start time and the work finish time of each worker are displayed, it is possible to manage working hours of workers.

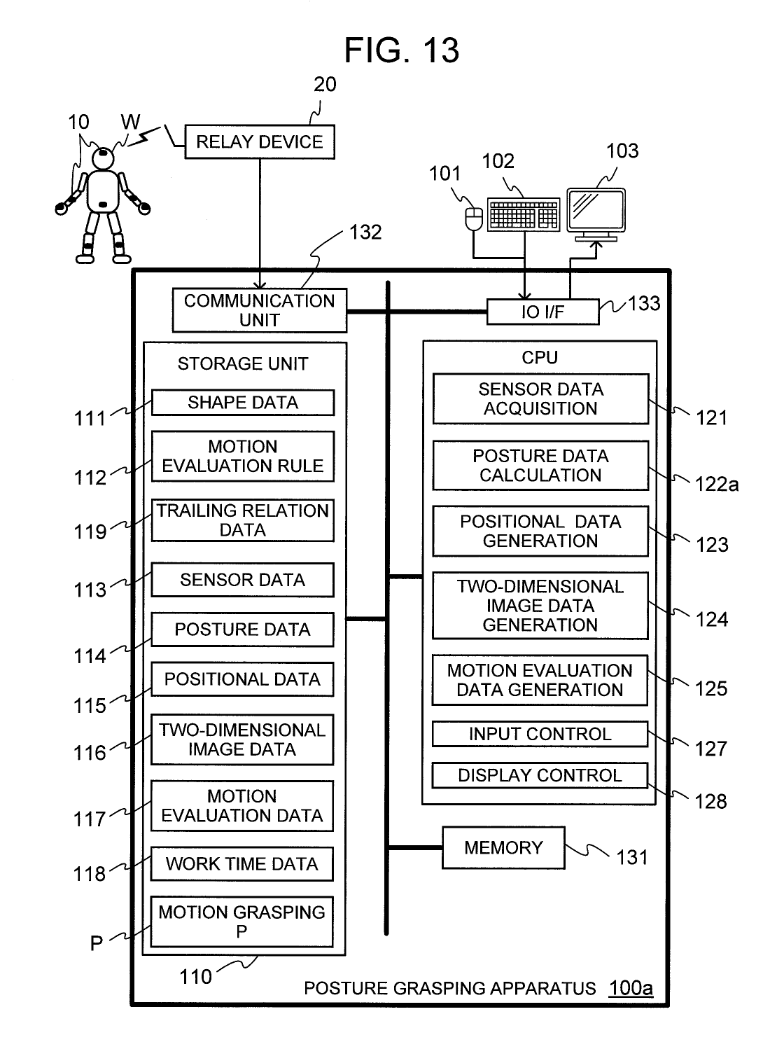

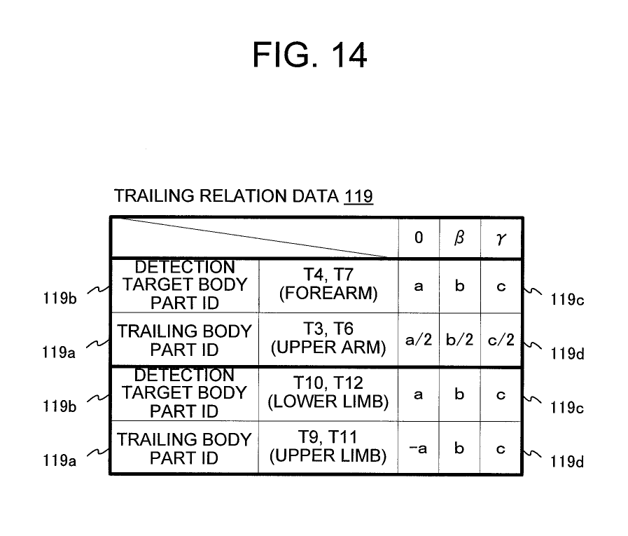

Next, a second embodiment of posture grasping system will be described referring to FIGS. 13 and 14.

In the first embodiment, the directional sensors 10 are attached to all body parts of a worker, and the posture data and the positional data are obtained on the basis of the sensor data from the directional sensors. On the other hand, in the present embodiment, a directional sensor is not used for some of the body parts of a worker, and posture data and positional data at such body parts are estimated on the sensor data from the directional sensors 10 attached to the other target body parts.

To this end, in the present embodiment, body parts each showing trailing movement along behind a movement of some body part are taken as trailing body parts, and a directional sensor is not attached to these trailing body parts. On the other hand, the other body parts are taken as detection target body parts, and directional sensors are attached to the detection target body parts. Further, as shown in FIG. 13, in the present embodiment, trailing relation data 119 indicating trailing relation between a posture of a trailing body part and a posture of a detection target body part that is trailed by that trailing body part are previously stored in the storage unit 110.

As shown in FIG. 14, the trailing relation data 119 are expressed in the form of a table. This table has: a trailing body part ID field 119a for storing an ID of a trailing body part; a detection target body part ID field 119b for storing an ID of a detection target body part that is trailed by the trailing body part; a reference displacement magnitude field 119c for storing respective rotation angles in the rotation directions α, β and γ of the detection target body part; and a trailing displacement magnitude field 119d for storing respective rotation angles in the rotation directions α, β and γ of the trailing body part. Each rotation angle stored in the trailing displacement magnitude field 119d is expressed by using the rotation angle stored in the reference displacement magnitude field 119c. Here, the detection target body part ID field 119b stores the IDs “T4, T7” of the forearms and the IDs “T10, T12” of the lower limbs. And, the trailing body part ID field 119a stores the IDs “T3, T6” of the upper arms as the trailing body parts of the forearms, and the IDs “T9, T11” of the upper limbs as the trailing body parts of the lower limbs. Thus, in this embodiment, a directional sensor 10 is not attached to the upper arms and the upper limbs as the trailing body parts of the worker.

For example, when a forearm is lifted, the upper arm also trails the motion of the forearm and is lifted in many cases. In that case, the displacement magnitude of the upper arm is often smaller than the displacement magnitude of the forearm. Thus, here, if the rotation angles in the rotation directions α, β and γ of the forearms T4 and T7 as the detection target body parts are respectively a, b and c, then the rotation angles in the rotation directions α, β and γ of the upper arms T3 and T6 as the trailing body parts are deemed to be a/2, b/2 and c/2 respectively. Further, when a knee is bent, the upper limb and the lower limb often displace by the same angle in the opposite directions to each other. Thus, here, if the rotation angle in the rotation direction α of the lower limbs T10 and T12 as the detection target body parts is a, the rotation angle in the rotation direction α of the upper limbs T9 and T11 as the trailing body parts is deemed to be −a. Further, as for the other rotation directions β and γ, it is substantially impossible because of the knee structure that an upper limb and the lower limb have different rotation angles. Thus, if the rotation angles in the rotation directions β and γ of the lower limbs T10 and T12 as the detection target body parts are respectively b and c, the rotation angles in the rotation directions β and γ of the upper limbs T9 and T11 as the trailing body parts are deemed to be respectively b and c also.

Next, operation of the posture grasping apparatus 100a of the present embodiment will be described.

Similarly to the step 10 in the first embodiment, in the present embodiment also, first the sensor data acquisition unit 121 of the posture grasping apparatus 100a receives data from the directional sensors 10, and stores the received data as the sensor data 113 in the storage unit 110.

Next, using the sensor data 113 stored in the storage unit 110, the posture data calculation unit 122a of the posture grasping apparatus 100a generates the posture data 114 and stores the generated posture data 114 in the storage unit 110. In so doing, as for data concerning the body parts included in the sensor data 113, the posture data calculation unit 122a performs processing similar to that in the step 20 of the first embodiment, to generate posture data of these body parts. Further, as for data of the body parts that are not included in the sensor data 113, i.e. data of the trailing body parts, the posture data calculation unit 122a refers to the trailing relation data 119 stored in the storage unit 110, to generate their posture data.

In detail, in the case where a trailing body part is the upper arm T3, the posture data calculation unit 122a first refers to the trailing relation data 119, to determine the forearm T4 as the detection target body part that is trailed by the posture of the upper arm T3, and obtains the posture data of the forearm T4. Then, the posture data calculation unit 122a refers to the trailing relation data 119 again, to grasp the relation between the posture data of the forearm T4 and the posture data of the upper arm T3, and obtains the posture data of the upper arm T3 on the basis of that relation. Similarly, also in the case where a trailing body part is the upper limb T9, the posture data of the upper limb T9 are obtained on the basis of the trailing relation with the lower limb T10.

When the posture data of all the body parts are obtained in this way, the obtained data are stored as the posture data 114 in the storage unit 110.

Thereafter, the processing in the steps 30-60 is performed similarly to the first embodiment.

As described above, in the present embodiment, it is possible to reduce the number of directional sensors 10 attached to a worker.

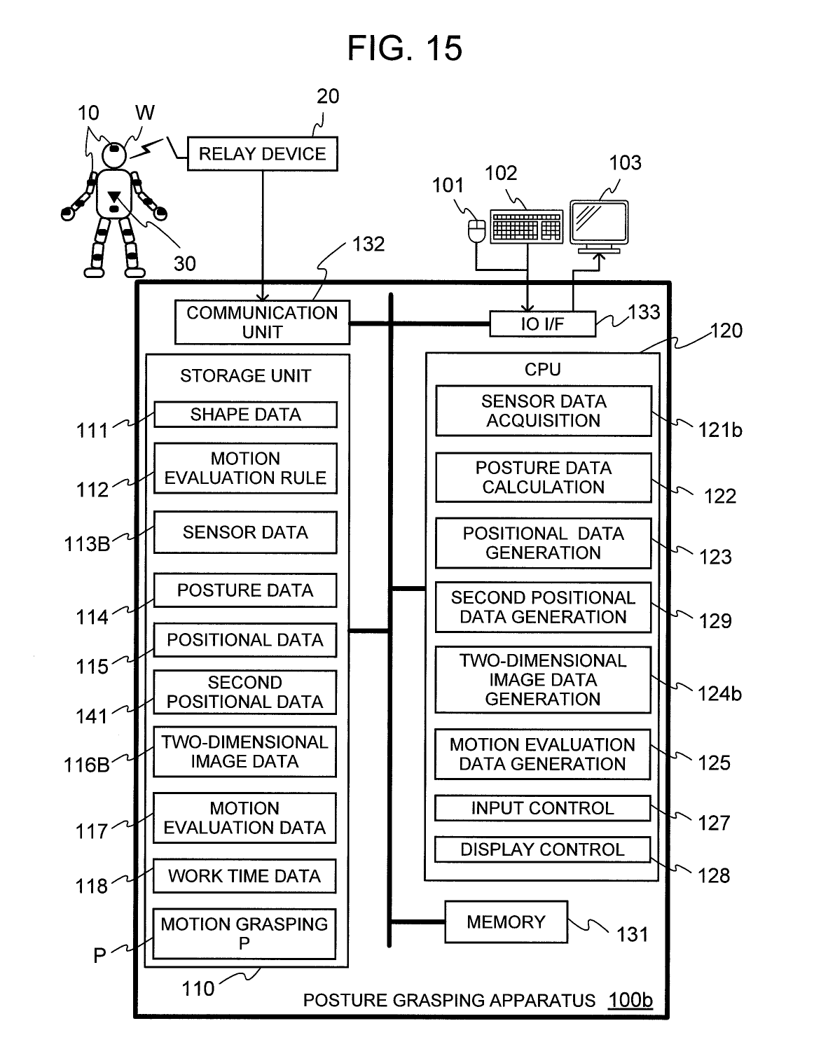

Next, a third embodiment of posture grasping system will be described referring to FIGS. 15-19.

As shown in FIG. 15, in the present embodiment, a location sensor 30 is attached to a worker as a target object, so that the location of the worker as well as the posture of the worker can be outputted.

Accordingly, the CPU 120 of the posture grasping apparatus 100b of the present embodiment functionally comprises (i.e. functions as), in addition to the functional units of the CPU 120 of the first embodiment: a second positional data generation unit 129 that generates second positional data indicating the location of the worker and positions of the body parts by using outputs from the location sensor 30 and the positional data generated by the positional data generation unit 123. Further, the sensor data acquisition unit 121b of the present embodiment acquires outputs from the directional sensors 10 similarly to the sensor data acquisition unit 121 of the first embodiment, and in addition acquires the outputs from the location sensor 30. Further, the two-dimensional image data generation unit 124b of the present embodiment does not use the positional data generated by the positional data generation unit 123 differently from the two-dimensional image data generation unit 124 of the first embodiment, but uses the above-mentioned second positional data, to generate two-dimensional image data. Each of the above-mentioned functional units 121b, 124b and 129 functions when the CPU 120 executes the motion grasping program P similarly to any other functional unit. The storage unit 110 stores the second positional data 141 generated by the second positional data generation unit 129 in the course of execution of the motion grasping program P.

The location sensor 30 of the present embodiment comprises a sensor for detecting a location, in addition to a power supply, a switch and a radio communication unit as in the directional sensor 10 described referring to FIG. 2. As the sensor for detecting a location, may be used a sensor that receives identification information from a plurality of transmitters arranged in a grid pattern in a floor, stairs and the like of a workshop and outputs location data on the basis of the received identification information. Or, a GPS receiver or the like may be used. In the above, the location sensor 30 and the directional sensors 10 have respective radio communication units. However, it is not necessary to have a radio communication unit. Instead of a radio communication unit, each of these sensors may be provided with a memory for storing the location data and the direction data, and the contents stored in the memory may be read by the posture grasping apparatus.

Next, operation of the posture grasping apparatus 100b of the present embodiment will be described referring to the flowchart shown in FIG. 18.

When the sensor data acquisition unit 121b of the posture grasping apparatus 100b receives data from the directional sensors 10 and the location sensor 30 through the communication unit 132, the sensor data acquisition unit 121b stores the data as the sensor data 113B in the storage unit 110 (S10b).

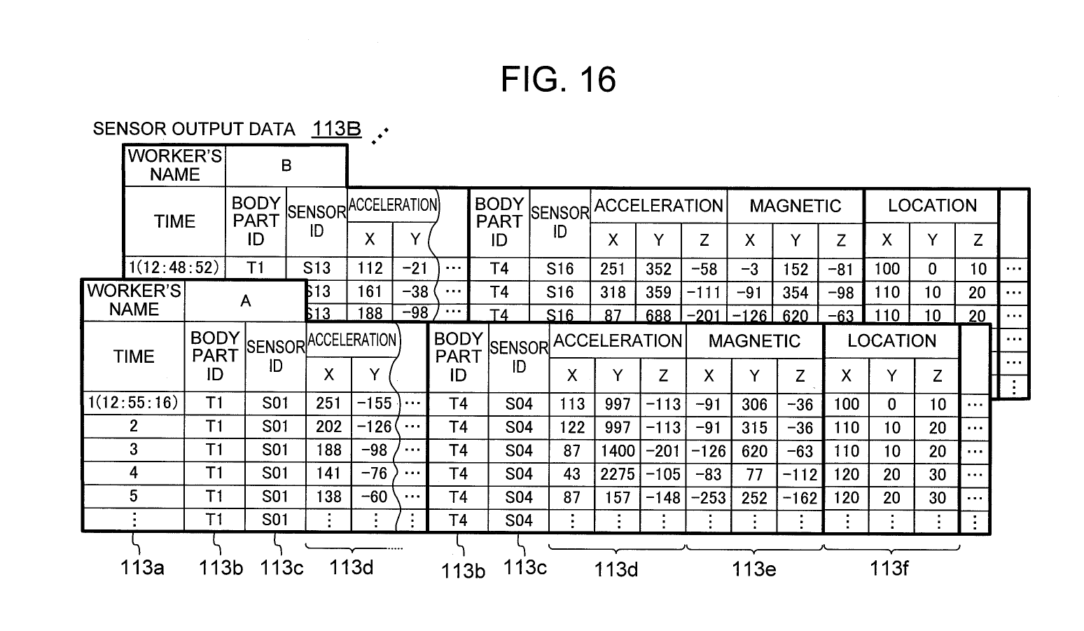

The sensor data 113B is expressed in the form of a table. As shown in FIG. 16, this table has, similarly to the sensor data 113 of the first embodiment: a time field 113a, a body part ID field 113b, a sensor ID field 113c, an acceleration sensor data field 113d, and a magnetic sensor data field 113e. In addition, this table has a location sensor data field 113f for storing X, Y and Z values from the location sensor 30. The X, Y and Z values from the location sensor 30 are values in the XYZ coordinate system having its origin at a specific location in a workshop. The directions of the X-, Y- and Z-axes of the XYZ coordinate system coincide respectively with the directions of the X-, Y- and Z-axes of the common coordinate system shown in FIG. 3.

Although, here, the data from the directional sensors 10 and the data from the location sensor 30 are stored in the same table, a table may be provided for each sensor and sensor data may be stored in the corresponding table. Further, although here outputs from the location sensor 30 are expressed in an orthogonal coordinate system, the outputs may be expressed in a cylindrical coordinate system, a spherical coordinate system or the like. Further, in the case where a sensor detecting a two-dimensional location is used as the location sensor 30, the column for the Y-axis (the axis in the vertical direction) in the location sensor data field 113f may be omitted. Further, although here a cycle for acquiring data from the directional sensor 10 coincides with a cycle for acquiring data from the location sensor 30, however data acquisition cycles for the sensors 10 and 30 may not be coincident. In that case, sometimes data from one type of sensor do not exist while data from the other type of sensor exist. In such a situation, it is favorable that missing data of the one type of sensor are interpolated by linear interpolation of anterior and posterior data to the missing data.

Next, similarly to the first embodiment, the posture data calculation unit 122 performs calculation processing of the posture data 114 (S20), and the positional data generation unit 123 performs processing of generating the positional data 115 (S30).

Next, the second positional data generation unit 129 generates the above-mentioned second positional data 141 (S35).

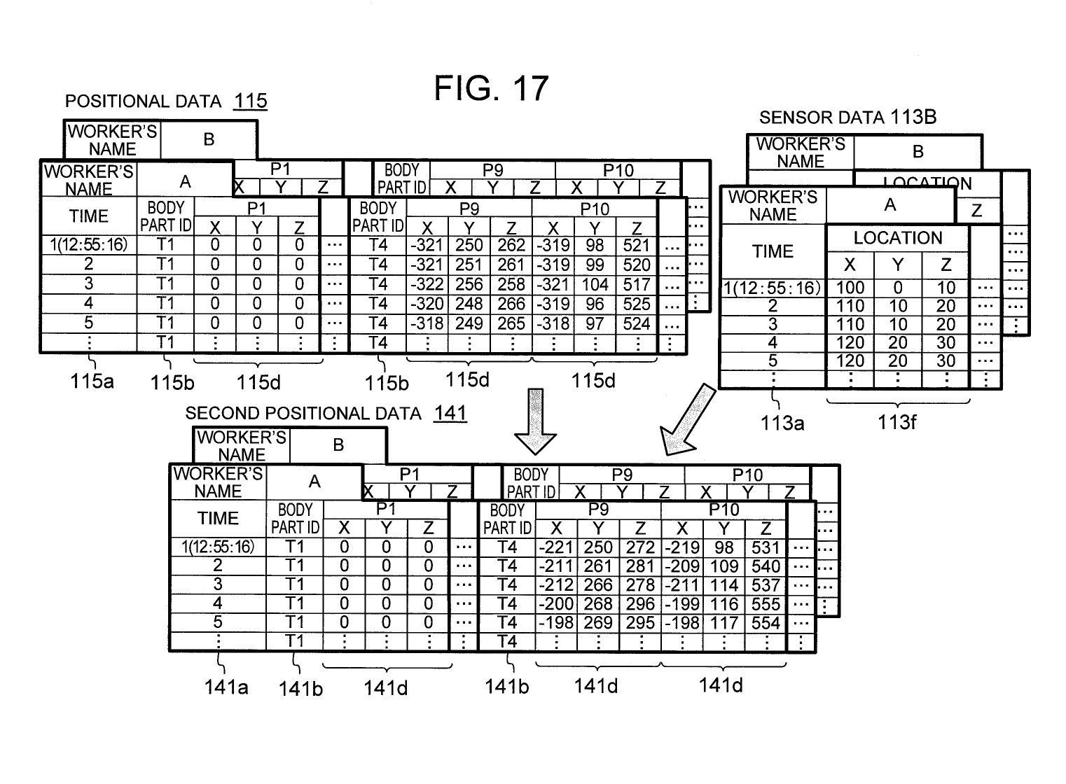

In detail, as shown in FIG. 17, the second positional data generation unit 129 adds data values stored in the coordinate data field 115d in the positional data 115 and data vales stored in the location sensor data field 113f in the sensor data 113b, to calculate second positional data values, and stores the obtained second positional data values in a coordinate data field 141d of the second positional data 141. In adding the data, two pieces of data of the same time and of the same body part of the same worker are added. The second positional data 141 have essentially the same data structure as the positional data 115, and have a time field 141a, a body part ID field 141b, in addition to the above-mentioned coordinate data field 141d. Although, here, the positional data 115 and the second positional data have the same data structure, the invention is not limited to this arrangement.

When the second positional data generation processing (S35) is finished, the two-dimensional image data generation unit 124b generates two-dimensional image data 114B by using the second positional data 141 and the shape data 111 (S40b) as described above. The method of generating the two-dimensional image data 114B is same as the method of generating the two-dimensional image data 114 by using the positional data 115 and the shape data 111 in the first embodiment.

Next, similarly to the first embodiment, motion evaluation data generation processing (S50) is performed and then output processing (S60b) is performed.

In this output processing (S60b), an output screen 150 such as shown in FIG. 12 is displayed on the display 103. Further, when the worker and the time are designated and additionally a location-shifting-type dynamic image is designated, then as shown in FIG. 19 the display control unit 128 displays, on the display 103, a schematic location-shifting-type dynamic screen 161 concerning the designated worker after the designated time by using the two-dimensional image data 114B.

Here, as shown in FIG. 19, in addition to the workers, articles 162 that are moved in the working process by the workers and fixed articles 163 that do not move may be displayed together, if such articles exist. In that case, it is necessary that directional sensors 10 and location sensors 30 are attached to these moving articles 162 and data on shapes of these articles have been previously stored in the storage unit 110. However, in the case of an article, there is no posture change of a plurality of parts, and thus it is sufficient to attach only one directional sensor 10 to such an article. Further, in that case, it is necessary that the shape data of the fixed articles 163 and coordinate values of specific points of the fixed articles 163 in a workshop coordinate system have been previously stored in the storage unit 110.

As described above, according to the present invention, not only postures of the body parts of the workers but also location shift of the workers and the articles can be grasped, and thus a behavior form of a worker can be grasped more effectively in comparison with the first and second embodiments.

In the above embodiments, the motion evaluation data 157a, 157b₁, and so on are obtained and displayed. These pieces of data may not be displayed, and simply the schematic dynamic screen 151, 161 of the worker may be displayed. Further, the output screen 150 displays the motion evaluation data 157a, 157b₁, and so on and the schematic dynamic screen 151 of the worker, and the like. However, it is possible to install a camera in the workshop, and a video image by the camera may be displayed synchronously with the dynamic screen 151, 161.

Further, in the above embodiments, after the workers finishes their work and the sensor data for the time period from start to finish of the work of each worker are obtained (S10), the posture data calculation processing (S20), the positional data generation processing (S30) and so on are performed. However, before the workers finish their work, the processing in and after the step 20 may be performed on the basis of already-acquired sensor data. Further, here, after the two-dimensional image data are generated with respect to all the body parts and over the whole time period (S40), the schematic dynamic screen 151 of a worker at and after a target time is displayed on the condition that the time specifying mark 159 is moved to the target time on the time scale 153 in the output processing (S60). However, it is possible that when the time specifying mark 159 is moved to a target time on the time scale 153, then at this point of time, two-dimensional data of the worker from the designated time are generated and the schematic dynamic screen 151 of the worker is displayed by using the sequentially-generated two-dimensional image data.

Further, in the above embodiments, as a directional sensor 10, one having an acceleration sensor 11 and a magnetic sensor 12 is used. However, in the case where a posture change of a target object does not substantially include rotation in the y direction, i.e. horizontal rotation, or in the case where it is not necessary to generate posture data considering horizontal rotation, the magnetic sensor 12 may be omitted and the posture data may be generated by using only the sensor data from the directional sensor 11.

1. A posture grasping apparatus for grasping a posture of a target part on a basis of output from a directional sensor that detects a direction in space and is attached to the target part among a plurality of target parts of a target object, comprising:
a shape data storage means that stores shape data of the target part to which the directional sensor is attached;
a sensor output acquisition means that acquires an output value from the directional sensor;
a posture data calculation means that uses the output value from the directional sensor to calculate posture data indicating a direction of the target part to which the directional sensor is attached with reference to reference axes directed in predetermined directions;
a positional data generation means that generates positional data on a position of the target part in space by using the target part's shape data stored in the shape data storage means and the target part's posture data calculated by the posture data calculation means, and by obtaining in space positional data of at least two representative points of the target part indicated in the shape data with reference to a connecting point with another target part connected with the target part in question;
a two-dimensional image generation means that generates two-dimensional image data indicating the target part by using the positional data, which are generated by the positional data generation means, on the position of the target part in space and the target part's shape data stored in the shape data storage means; and
an output means that outputs a two-dimensional image of the target part on a basis of the target parts' two-dimensional image data generated by the two-dimensional image generation means.

2. A posture grasping apparatus of claim 1, wherein:
the positional data generation means makes connecting points of two target parts connected with each other among the plurality of target parts have same value of positional data.

3. A posture grasping apparatus of claim 1, wherein:
the directional sensors are attached to all of the plurality of the target parts of the target object;
the shape data storage means stores shape data of all of the plurality of the target parts of the target object; and
the output means outputs two-dimensional images of all of the plurality of target parts of the target object.

4. A posture grasping apparatus of claim 1, wherein:
among the plurality of target parts of the target object, directional sensors are attached to detection target parts i.e. target parts other than trailing target parts which show a movement of trailing a movement of another target part;
the posture grasping apparatus comprises a trailing relation storage means that stores trailing relations between postures of the trailing target parts and postures of the detection target parts that are trailed by the trailing target parts;
the shape data storage means stores all shape data of the plurality of target parts of the target object;
the posture data calculation means calculates posture data of the detection target parts, and thereafter calculates posture data of the trailing target parts by using the posture data of the detection target parts and the trailing relations between the detection target parts and the trailing target parts; and
the output means outputs all two-dimensional images of the plurality of target parts of the target object.

5. A posture grasping apparatus of claim 3, wherein:
a connecting point of one target part to which the directional sensor is attached with another target part connected with the one target part among the plurality of target parts of the target object is taken as a reference position in space, and positional data of the one target point in space are obtained, and thereafter positional data of another target part connected to the target part whose positional data have been obtained are obtained sequentially.

6. A posture grasping apparatus of claim 1, wherein:
the sensor output acquisition means acquires output values from the directional sensor on a time series basis; and
the output means outputs two-dimensional images of the target part in time series order.

7. A posture grasping apparatus claim 1, wherein:
the posture grasping apparatus comprises:
an evaluation rule storage means that stores a motion evaluation rule i.e. a relation between a magnitude of displacement of the target part from a reference posture and a motion level of the target part, for each displacement mode of the target part; and
a motion level calculation means that uses the motion evaluation rule concerning the target part to obtain a motion level of the target part from the magnitude of displacement of the target part with respect to a displacement mode to which the motion evaluation rule is applied; and
the output means outputs the target part's motion level obtained by the motion level calculation means.

8. A posture grasping apparatus of claim 6, wherein:
the posture grasping apparatus comprises:
an evaluation rule storage means that stores a motion evaluation rule i.e. a relation between a magnitude of displacement of the target part from a reference posture and a motion level of the target part, for each displacement mode of the target part; and
a motion level calculation means that uses the motion evaluation rule concerning the target part to obtain a motion level of the target part from the magnitude of displacement of the target part with respect to a displacement mode to which the motion evaluation rule is applied; and
the output means outputs on a time series basis the target part's motion level obtained by the motion level calculation means.

9. A posture grasping apparatus of claim 8, wherein:
the posture grasping apparatus comprises a posture display time receiving means that receives designation of any time among times of the target part's motion levels outputted by the output means on the time series basis; and
when the posture display time receiving means receives the designation of time, the output means outputs on a time series basis two-dimensional images of the target part after the designated time.

10. A posture grasping apparatus of claim 6, wherein:
the output means outputs an acquisition start time and an acquisition finish time of acquisition of output values from the directional sensor.

11. A posture grasping apparatus of claim 1, wherein:
the sensor output acquisition means acquires an output value from a location sensor attached to the target object;
the posture grasping apparatus comprises a second positional data generation means that generates second positional data of the target part by moving the positional data of the target part generated by the positional data generation means, depending on the output value from the location sensor; and
the two-dimensional image generation means generates two-dimensional image data indicating the target part, by using the second positional data of the target part generated by the second positional data generation means, instead of the positional data of the target part generated by the positional data generation means.

12. A posture grasping system comprising:
a posture grasping apparatus of claim 1; and the directional sensor.

13. A posture grasping system of claim 12, wherein:
the directional sensor comprises: an acceleration sensor; a magnetic sensor; and a radio communication unit for wirelessly transmitting outputs from the acceleration sensor and the magnetic sensor.

14. A posture grasping program for grasping a posture of a target part among a plurality of target parts of a target object, on a basis of an output from a directional sensor for detecting a direction in space, the directional sensor being attached to the target part in question, wherein:
the posture grasping program makes a computer execute:
a sensor output acquisition step, in which a communication means of the computer acquires an output value from the directional sensor;
a posture data calculation step, in which posture data indicating a direction of the target part, to which the directional sensor is attached, with reference to reference axes that are directed in previously-determined directions are calculated by using the output value from the directional sensor;
a positional data generation step, in which positional data of the target part in space are generated by using shape data of the target part previously stored in a storage unit of the computer and the posture data of the target part calculated in the posture data calculation step, and by obtaining positional data in space of at least two representative points in the target part indicated in the shape data, with reference to a connecting point with another target part connected with the target part in question;
a two-dimensional image generation step, in which two-dimensional image data indicating the target part are generated by using the positional data in space of the target part, which are generated in the positional data generation step, and the shape data of the target part stored in the storage unit; and
an output step, in which a two-dimensional image of the target part is outputted on a basis of the two-dimensional image data of the target part generated in the two-dimensional image generation step.

15. A posture grasping method for grasping a posture of at least one target part among a plurality of target parts of a target object, wherein:
a directional sensor for detecting a direction in space is attached to the at least one target part; and
a computer execute:
a sensor output acquisition step, in which a communication means of the computer acquires an output value from the directional sensor;
a posture data calculation step, in which posture data indicating a direction of the target part, to which the directional sensor is attached, with reference to reference axes that are directed in previously-determined directions are calculated by using the output value from the directional sensor;
a positional data generation step, in which positional data of the target part in space are generated by using shape data of the target part previously stored in a storage unit of the computer and the posture data of the target part calculated in the posture data calculation step, and by obtaining positional data in space of at least two representative points in the target part indicated in the shape data, with reference to a connecting point with another target part connected with the target part in question;
a two-dimensional image generation step, in which two-dimensional image data indicating the target part are generated by using the positional data in space of the target part, which are generated in the positional data generation step, and the shape data of the target part stored in the storage unit; and
an output step, in which a two-dimensional image of the target part is outputted on a basis of the two-dimensional image data of the target part generated in the two-dimensional image generation step.
